(19) United States
(12) Patent Application Publication
(10) Pub. No.: US 2024/0269492 A1
(43) Pub. Date: Aug. 15, 2024

(54) A PERSONAL NECK-WEARABLE AIR PURIFICATION DEVICE

(71) Applicant: COSMOAESTHETICS PTY LTD, Kew (AU)

(72) Inventor: Masoud JAFARZADEH, Kew (AU)

(21) Appl. No.: 18/566,820

(22) PCT Filed: Jun. 24, 2022
(86) PCT No.: PCT/AU2022/050642

§ 371 Date: Dec. 4, 2023

(30) Foreign Application Priority Data
Jul. 15, 2021 (AU) 2021902169

(51) Int. Cl. A62B 18/00; A61H 23/02; A62B 18/08; A62B 23/02

(52) U.S. Cl. CPC A62B 18/003; A61H 23/02; A62B 18/08; A62B 23/02; A61H 2201/107; A61H 2201/5007; A61H 2205/04

(57) ABSTRACT

A personal neck-wearable device is configured to direct a curtain of protective air around the face of a wearer in use to prevent or reduce inhalation of airborne particles and pathogens. The air may be purified using filtration. In an embodiment, the air may be ionised using high voltage to ionise the air molecules with negative ions (anions) having one or more extra electrons, thereby negatively charging the air molecules. The curtain of ionised air molecules surrounds the face of the user and attracts airborne air particles by electrostatic attraction. These particles are then attracted to the nearest surface away from the mouth and nose to thereby reduce the likelihood of inhalation.

### FIELD OF THE INVENTION

This invention relates generally to personal neck-wearable device configured to direct a curtain of protective air around the face of a wearer in use.

### SUMMARY OF THE DISCLOSURE

There is provided herein a personal neck-wearable device configured to direct a curtain of protective air around the face of a wearer in use to prevent or reduce inhalation of airborne particles and pathogens.

The air may be purified using filtration. In an embodiment, the air may be ionised using high voltage to ionise the air molecules with negative ions (anions) having one or more extra electrons, thereby negatively charging the air molecules.

The curtain of ionised air molecules surrounds the face of the user and attracts airborne air particles by electrostatic attraction. These particles are then attracted to the nearest surface away from the mouth and nose to thereby reduce the likelihood of inhalation.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIGS. 1 to 3 show a personal neck-wearable air purification device in accordance with an embodiment.

FIG. 4 shows exemplary control circuitry of the device;

FIG. 5 shows a front perspective view of the device;

FIG. 6 shows a top view of the device;

FIG. 7 shows an internal perspective view of the device; and

FIGS. 8 and 9 show a slot in filtration element in accordance with an embodiment.

### DESCRIPTION OF EMBODIMENTS

A personal neck-wearable device 100 defines an air circuit 105 therethrough between at least one intake 106 and vent 107. The air circuit 105 comprises a fan 108 to force air through the air circuit 105.

In the manner shown in FIGS. 2 and 3, the vent 107 is located to direct a curtain 110 of protective air around the face of the wearer to shield against inhalation of airborne particles and pathogens. In embodiments, the air flowing through the air circuit 105 is filtered.

The device 100 may comprises an air ioniser 109 which generate negative ions to ionise air passing through the air circuit 105. A boost converter 120 may convert approximately 12V from battery supply 116 to approximately 8 kV for the ioniser 109. The ionised air curtain 110 attracts airborne particles by electrostatic attraction.

In embodiments, an exterior surface of the device 100 may comprise a grounding plate having a different electrostatic potential compared to that of the ionised particles to attract airborne particles which have been charged by the ionised air curtain 110 away from the face of the wearer. The grounding plate may locate across a top surface of the device 100.

The control circuitry 104 may comprise a controller 112 for controlling the operation of the device 100. The controller 112 may comprise a microprocessor 113 in operable communication with a memory device 114. The memory device 114 may store digital data including computer program code instructions. In use, the microprocessor 113 fetches these computer program code instructions and associated data from the memory device 114 for interpretation and execution of the operational functionality described herein. The controller 112 may comprise an I/O interface 115 for interfacing with the various components of the device 100.

The circuitry 104 may comprises a rechargeable battery supply 116 which may be periodically recharged by recharging circuitry 117. The recharging circuitry 117 may interface a recharging port exposed by the device. The recharging port may be a USB compatible. The controller 112 may draw power from the battery supply 116 in use.

The controller 112 may interface a wireless data interface 119 including for interoperability with a software application executing on an electronic device such as a smartphone device. The software application executing on a smartphone device may be used to control the operational functionality of the controller 112 in use. The controller 112 may be configured to receive operational instructions from the electronic device via the wireless data interface 119 and control the air circuit 105 accordingly, such as the operation of the fan 108 and/or the ioniser 109. In embodiments, the air circuit comprises various sensors to monitor air pollutant levels and wherein the controller transmits air pollution statistics to the electronic device via the wireless interface.

The device 100 according to the embodiments shown may have a substantially horseshoe-shaped housing 130 which is worn across the back of the neck in the manner shown in FIGS. 2 and 3.

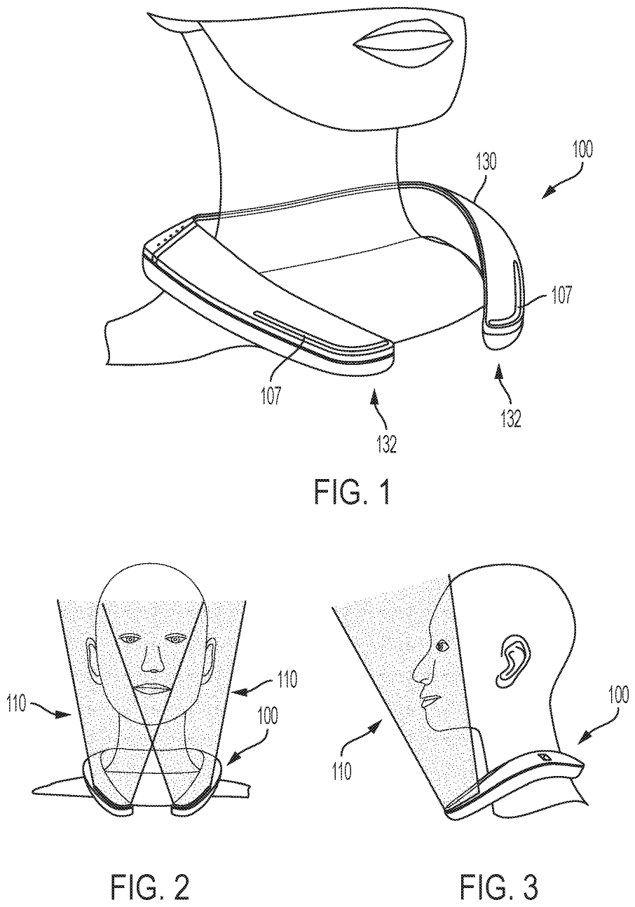

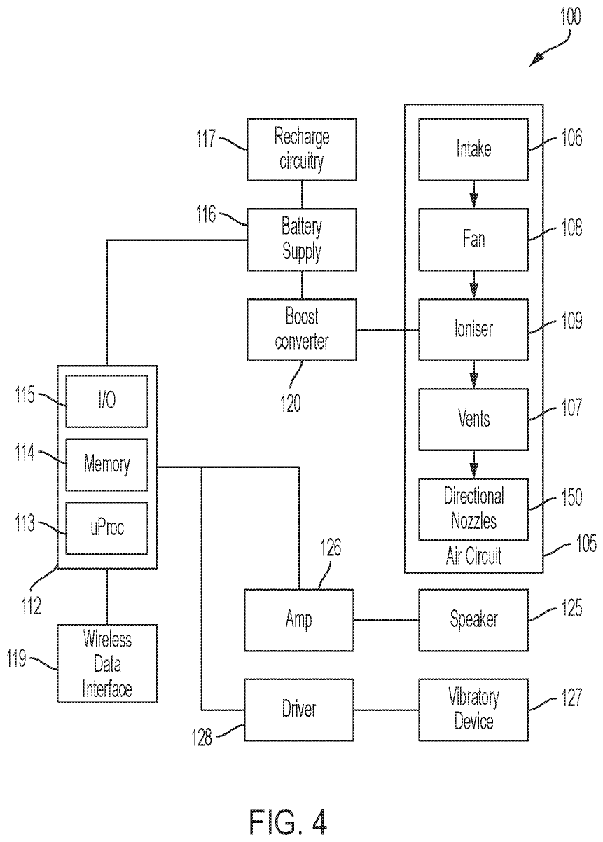

Specifically, with reference to FIG. 6, the housing 130 may comprise a central portion 131 and side arms 132. Distal ends of the arms 132 may form a gap 133 therebetween. The side arms 132 and central portion 131 surround an area 134 therebetween which fits round the lower neck in the manner shown in FIG. 1.

In embodiments, the housing 130 may be opened to widen the gap 133 for the neck when putting on and taking of the device. The central portion 131 may comprise a hinge mechanism 135 allowing the arms 132 to move apart. The hinge mechanism 135 may be biased towards the closed position so that the device 100 closes around the neck. Electrical wires may run across the hinge mechanism 135 to electrically connect each arm 132. In alternative embodiments, the hinge mechanism 135 has electrical connections which close an electrical circuit when the arms 132 move together.

As is shown in FIG. 3, the central region 131 may be worn substantially horizontally across the back of the neck whereas the arms 132 may curve downwardly to contour down across the front of the neck and shoulders.

As is shown in FIG. 6, the area 134 may be generally of an inverted teardrop shape in cross-section wherein the area 134 may have a generally constant radius towards the rear of the device 100 (i.e. generally circular).

The upper surface 136 of the housing 130 may be generally planar and may slope outwardly slightly.

Each arm 132 may define a respective air circuit 105. In this regard, an upper surface of each arm 132 may comprise respective air intake 106.

The air intakes 136 may be shaped to direct air towards distal ends of the arms 132 and therefore may comprise a rounded rearward aspect which interfaces a rounded channel portion thereunderneath going forward towards the distal ends of the arms 132.

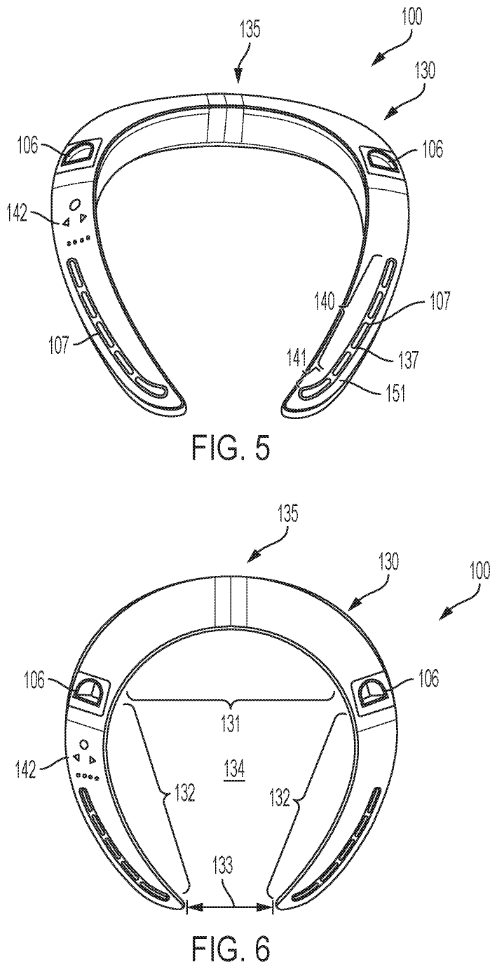

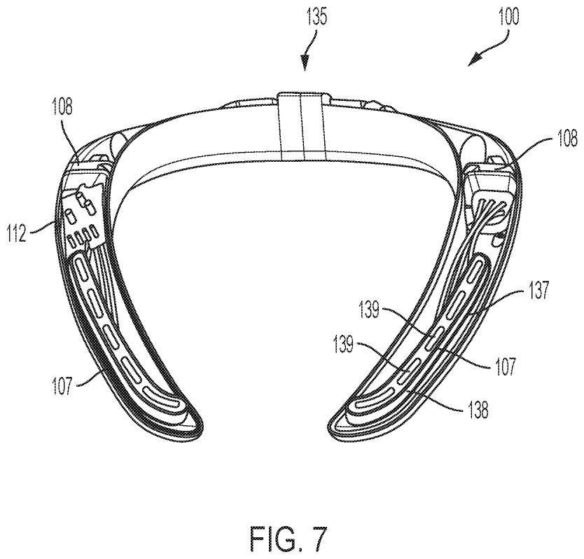

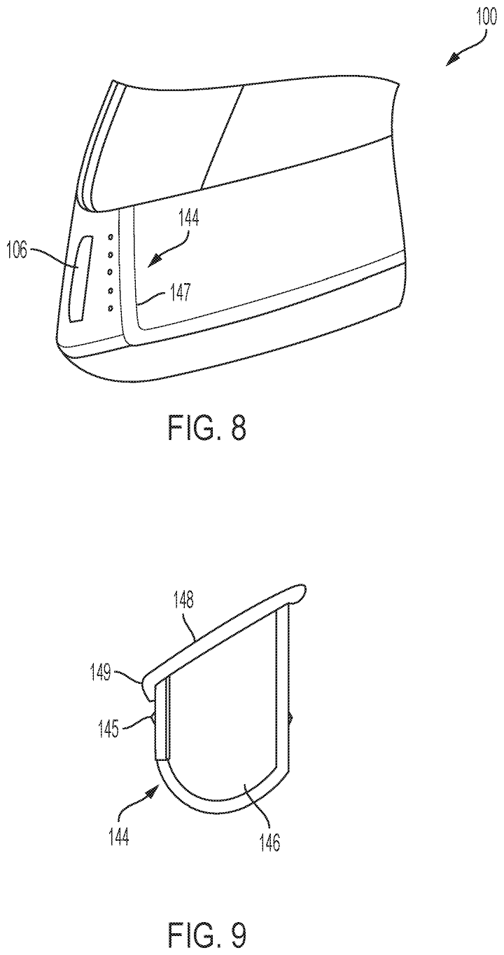

As is shown in FIG. 5, each vent 107 may be elongate, positioned at a distal ends of a respective arm 132 and be aligned along the length of the respective arm 132. As such, the vents 107 may be suitably shaped and configured to direct narrow blades of air around the face. The blades 110 of air may have an arc width which may extend up to 180° in embodiments.

The vent 107 may comprise an air guide piece 137 which fits through a shaped slot 151 through an upper surface of the housing 132 of each arm 132.

The guide piece 137 may comprise a bevelled edge 138 interfacing the shaped slot 151 through the upper surface 136 of the housing 130 in an airtight manner. Furthermore, the guide piece 137 may comprise a plurality of openings 139 therethrough through which air escapes.

In embodiments, directional nozzles 150 may interface the vents 107. In embodiments, electromechanical actuators may interface the directional nozzles 150 under control of the controller 112.

The guide piece 137 directs air substantially upwardly and generally orthogonally with respect to each arm 132 in the manner shown in FIGS. 2 and 3, thereby creating a curtain of air 141 around the face of the wearer.

The location and orientation of the guide pieces 139 preferably directs the curtain of air in front of the face but away from the face so that the face is not blown by the curtain of air.

Furthermore, each guide piece 137 may comprise a generally straight rearward section 140 and an inwardly transitioning terminus 141 towards the distal end of the arm 132 which shapes the curtain of air 110. In this way, the curtain of air 144 may be essentially defined by two separate blades of air 110 which are spaced apart on either side of the face, each blade 110 having a rear generally straight cross section which goes adjacent the side of the face and a forward inward curvature which curves in towards and in front the nose and mouth of the wearer.

The device 100 may comprise a user control interface 142 comprising controls for controlling the operation of the air circuit 105, including the velocity of air and operation of the ioniser 109.

In embodiments, the device 100 may comprise a vibratory massage device 127 to gently massage the neck. The vibratory massage device 127 may be driven by a driver 128 controlled by the controller 112 to provide different programs of vibration, including vibration patterns, frequency and intensity.

In embodiments, the device 100 may comprise a speaker 125 driven by an amplifier 126 and controlled by the controller 112 to play audio. With reference to FIG. 1, the speakers 125 may be located on inner surfaces of the pods 103. The device 100 may be configured for playing music from a smartphone device via a Bluetooth wireless data interface 119. The device may comprise 3.5 mm headphone jack for use instead of the speakers 125 which disables the operation of the speakers 125 when a headphone plug is plugged thereinto.

The control interface 142 may further control the operation of the massage device 127 (i.e., the amplitude and/or frequency) and/or the speaker 125 (i.e., the volume, and track controls).

The control interface 142 may further comprise operational indicating LEDs which may indicate various operational modes of the device 100.

As shown in FIG. 10, the device may comprise a slot-in HEPA filtration component 144. The component 144 may comprise an outer frame 145 of plastic which supports HEPA filtration material 146 therein. The frame 145 may be narrow to allow the component 144 to slot in sideways into a corresponding slot 147 of the housing 130. As is shown in FIG. 10, the filtration component 144 may slot into the air circuit 105 between the intake 106 and the vent 107, preferably prior the fan 108.

The frame 145 may comprise an upper section 148 which may be generally straight so as to conform with the planar upper surface of the housing 130. The upper section 148 may comprise an edge 149 under which a fingernail may be engaged to pull the filtration component 144 from the housing 130.

The ioniser 109 may be located within the air circuit 105 to ionise air before the air is filtered through the slot-in HEPA filtration components 144 to aid entrapment of negatively charged particles within the filtration material 106.

In embodiments, the device 100 directs parallel narrow blades 110 of air towards the face of the user. These blades 110 may comprise an innermost blade comprising non-ionised air and the outermost blade comprising ionised air.

Whereas the outermost ionised air blade 110 may electrostatically charge particles which may then be attracted to adjacent surfaces, including the face, the innermost blade of non-ionised air deflects such charged particles away from the nose and mouth of the user, thereby preventing the charge particles from adhering thereto.

In one embodiment, one nozzle 107 of one arm 132 may direct the outermost ionised air blade and the other nozzle 107 of the other arm 132 may direct the innermost non-ionised air blade.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. A personal neck-wearable device defining an air circuit therethrough between an intake and a vent, the air circuit comprising a fan to force air through the air circuit and wherein the vent is located to direct a curtain of air around a face of a wearer in use.

2. The device as claimed in claim 1, wherein the vent is elongate so that the curtain of air is formed by at least one blade of air.

3. The device as claimed in claim 2, wherein the vent is located to direct a curtain of air in front of the face of the wearer in use but away from the face of the wearer in use so that the face is not blown by the curtain of air.

4. The device as claimed in claim 2, wherein the at least one blade of air goes through 180° around the face.

5. The device as claimed in claim 1, wherein the device comprises an air ioniser to ionise air passing through the air circuit.

6. The device as claimed in claim 5, wherein the device comprises electronic circuitry comprising a boost converter to boost voltage from an internal battery supply for the ioniser.

7. The device as claimed in claim 6, further comprising a controller controlling the operation of the air circuit and wherein the controller is configured to control operation of the ioniser.

8. The device as claimed in claim 5, wherein an exterior surface of the device comprises a grounding plate to attract airborne particles which have been charged by ionised air from the ioniser.

9. The device as claimed in claim 1, further comprising a controller controlling the operation of the air circuit.

10. The device as claimed in claim 9, wherein the controller is configured to control air velocity.

11. The device as claimed in claim 1, further comprising an electronic device in operable communication with the device via a wireless interface and wherein the device comprises a controller configured to receive operational instructions from the electronic device and control the air circuit accordingly.

12. The device as claimed in claim 11, wherein the air circuit comprises a sensor to monitor air pollutant levels and wherein the controller transmits air pollution statistics measured by the sensor to the electronic device via the wireless interface.

13. The device as claimed in claim 1, wherein the device has a substantially horseshoe-shaped housing defining side arms.

14. The device as claimed in claim 13, further comprising a hinge mechanism which allows the arms to move apart.

15. The device as claimed in claim 13, wherein the side arms curve downwardly with respect to a central portion of the housing therebetween.

16. The device as claimed in claim 15, wherein the central portion has a generally semicircular shape.

17. The device as claimed in claim 13, wherein each arm contains a respective air circuit.

18. The device as claimed in claim 17, wherein each arm comprises a respective air intake for each air circuit.

19. The device as claimed in claim 18, wherein each intake is located on an upper surface of each respective arm.

20. The device as claimed in claim 18, wherein the air intake is shaped to direct air towards a distal end of each arm.

21. The device as claimed in claim 17, wherein each arm comprises a respective vent.

22. The device as claimed in claim 21, wherein each vent is elongate so that the curtain of air is formed by two blades of air formed by respective vents.

23. The device as claimed in claim 21, wherein each vent comprises a generally straight rear section and an inwardly transitioning front terminus towards a distal end of a respective arm.

24. The device as claimed in claim 22, wherein each vent is positioned at a distal end of a respective arm and aligned along a length of the respective arm.

25. The device as claimed in claim 21, wherein each vent comprises an air guide piece which fits through a respective shaped slot through an upper surface of the housing.

26. The device as claimed in claim 25, wherein each guide piece comprises a bevelled edge to interface the respective slot in an airtight manner.

27. The device as claimed in claim 25, wherein the guide piece comprises a plurality of openings therethrough.

28. The device as claimed in claim 1, wherein the air through the air circuit is filtered.

29. The device as claimed in claim 28, wherein the housing comprises a slot for inserting a filtration component into the air circuit.

30. The device as claimed in claim 29, wherein the device comprises an air ioniser to ionise air passing through the air circuit and wherein the ioniser is located within the air circuit to ionise air before the air is filtered through the filtration component.

31. The device as claimed in claim 1, further comprising a directional nozzle interfacing the vent

32. The device as claimed in claim 31, further comprising electromechanical actuators which interface the directional nozzles under control of the controller.

33. The device as claimed in claim 1, wherein the device directs overlapping blades of air towards the face of the user.

34. The device as claimed in claim 33, wherein the device comprises an air ioniser to ionise air passing through the air circuit wherein the overlapping blades of air comprises an innermost blade comprising non-ionised air and the outermost blade comprising ionised air.

35. The device as claimed in claim 34, wherein the device has a substantially horseshoe-shaped housing defining side arms wherein one nozzle of one arm directs the outermost ionised air blade and the other nozzle of the other arm directs the innermost non-ionised air blade.
